(19) United States
(12) Patent Application Publication
(10) Pub. No.: US 2002/0115955 A1
(43) Pub. Date: Aug. 22, 2002

(54) Surgical sock

(76) Inventor: Danforth DeSena, Cape Elizabeth, ME (US)

Correspondence Address: Chris A. Caseiro, Pierce Atwood, One Monument Square, Portland, ME, 04101, US

(21) Appl. No.: 09/785,326

(22) Filed: Feb. 16, 2001

(51) Int. Cl. A61F 13/00

(52) U.S. Cl. 602/61; 602/60

(57) ABSTRACT

A surgical sock for covering a bandaged foot. The sock includes a forefoot region, a foot region, and an ankle region. The forefoot region, the foot region, or a combination of the two has a dimension greater than that of the ankle region in order to accommodate a bandaged foot without undue constriction or excessive wrinkling. The ankle region may have greater elasticity than the other regions so that it expands enough to accept the bandaged foot while retaining enough grasping strength to keep it in place about the ankle. The sock is fabricated with a hydrophobic fiber, a bacteriostatic fabric, or both to aid in moisture wicking, to minimize the chance of post-operative infection, and to minimize overheating.

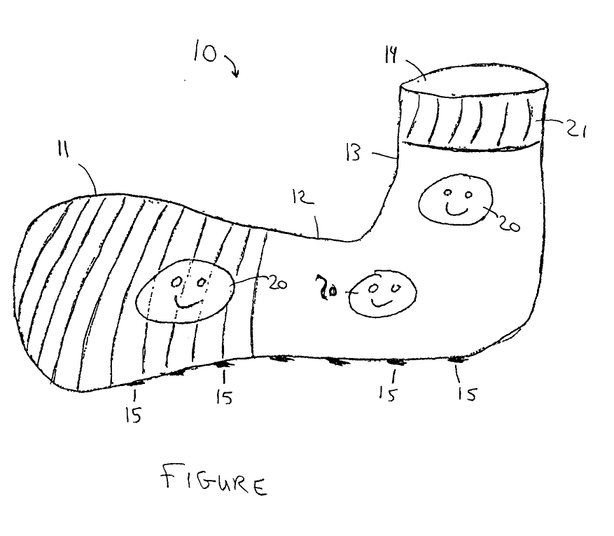

### BACKGROUND OF THE INVENTION

1. Field of the Invention

The present invention relates to cover wear placed over the foot after surgical procedures. More particularly, the present invention relates to post-surgery surgical socks.

2. Description of the Prior Art

Presently, after a surgical procedure has been performed on a foot, the area affected is covered with a removable bandage. In order to minimize detrimental environmental effects on the bandage that is adjacent to the surgical wound, a sock of some type is placed on the foot including over the affected area. On some occasions, the sock may simply be a conventional sock, such as an athletic tube sock of substantially uniform diameter. More often, the healthcare provider modifies a section of cotton tubular gauze. Specifically, the gauze is cut, one portion is pulled over the foot and above the ankle, and the remainder is rotated about the toe region in an attempt to seal off the open end of the cut tubular gauze. Any excess is folded back over the foot and lower leg to create a double layer sock-like covering. This process takes considerable time and effort on the part of the healthcare provider.

As might be expected, there is inherent potential for inconsistency in the application of this customized external surgical sock. There are additional deficiencies in such surgical socks. First, the tubular gauze is of uniform diameter and elasticity, just like the conventional sock. As a result, when one part is large enough to fit over the surgical bandage, other adjacent parts are too loose at the ankle and the patient is unable to keep the sock in a fixed position over the ankle. Second, the wrapping of the gauze creates a plurality of layers that wrinkle, thereby causing irritation to the skin. Third, when the remainder of the gauze is rotated and folded back over the toes, it creates an irritating mass in that region. In addition to being uncomfortable, the mass may cause excessive pressure in that area including the wound. That pressure may be harmful to the purpose of the surgical procedure.

As a result of the desire to improve patients' condition after foot surgery, several covering devices have been made the subject of patents. For example, U.S. Pat. No. 5,697,106 issued to Baker et al. describes a waterproof cover including an elastic means to ensure that the cover remains in place. U.S. Pat. No. 5,083,557 issued to Lennon et al. describes a podiatry boot for placement over a cast or dressing. The boot includes a fastening component to facilitate entry into and exit out of the boot. U.S. Pat. Nos. 5,016,648 and 4,530,350 issued to Brown et al. describe similar protective boots. U.S. Pat. No. 4,021,860 issued to Swallow et al. describes a therapeutic stocking including means to prevent slipping. U.S. Pat. No. 1,980,486 issued to King et al. describes a waterproof protective foot covering with strapping to secure the ankle region of the covering about the ankle.

The prior devices identified above fail to describe a simple effective sock that can be used easily by the patient, that comfortably conforms about the bandaged region of the foot, that is retained in position about the ankle, and that fits within the commonly used surgical shoes. The coverings of the prior art either fail to address the fact that the bandaged foot area is typically much larger around than the ankle region or they include difficult-to-use clasping devices, such as that taught by King et al.

Therefore, what is needed is a covering suitable for use over a bandaged foot. Further, what is needed is such a surgical sock that is wide enough to cover loosely the foot region that includes the bandage. Yet further, what is needed is such a surgical sock that comfortably and snugly fits about the ankle in a manner that prevents the sock from slipping downward. Still further, what is needed is such a surgical sock that is relatively simple to use and that is relatively inexpensive.

### SUMMARY OF THE INVENTION

It is an object of the present invention to provide a covering suitable for use over a bandaged foot. It is also an object of the present invention to provide a surgical sock that is wide enough to cover snugly the foot region that includes the bandage. Another object of the present invention to provide such a surgical sock that comfortably and snugly fits about the ankle in a manner that prevents the sock from slipping downward. Further, it is an object of the present invention to provide a surgical sock that is relatively simple to use and that is relatively inexpensive.

These and other objects of the present invention are achieved by forming a surgical sock having a first wider region associated with the foot and a second narrower region associated with the ankle. The wider region is located at the front of the foot, particularly in the toe region where surgical procedures commonly occur. As a result, the sock does not constrict the bandaged area in an uncomfortable way. Alternatively, an array of sock types with differing locations for the wider region may be developed and applied dependent upon where the wound bandage is located. The underside of the sock may include anti-slip components to reduce the chance of a post-operative fall.

The sock may be fabricated of any type of relatively elastic material. The sock may be designed with greater elasticity in the ankle region and less elasticity in the foot region so that the bandaged foot may be inserted into the opening near the ankle region. Greater elasticity at that location allows for increased widening of the opening for passage of the bandaged foot therein. The foot region is fabricated of a material that is relatively wrinkle free and that has no way to knot at the toe region. The material used to make the sock is breathable in that it does not encourage moisture entrapment and heating. In that way, it minimizes the possibility of enhancing the environment for infection development.

These and other advantages of the present invention will become apparent upon review of the following description, the accompanying drawing, and the appended claims.

### BRIEF DESCRIPTION OF THE DRAWING

The FIGURE is a side view of the preferred embodiment of the surgical sock of the present invention.

### DESCRIPTION OF THE INVENTION

A surgical sock 10 of the present invention is shown in the FIGURE. It is formed of a suitably elastic material, such as elasticized cofton, polyester, polyester fleece, Spandex™, Lycra™, or nylon, but is not limited thereto. The material for the sock 10 is optionally made with a bacteriostatic fabric to reduce the chance of post-operative infection. It may also optionally be made with a hydrophobic fiber to wick perspiration away from the foot. Such a fiber is of particular importance to maximize wound healing since most wound dressings are relatively bulky and heat retaining. The size of the surgical sock 10 is only limited by the size of the bandaged foot to be covered and it is contemplated that an array of sizes of the sock 10 would be made available to a healthcare provider. The sock 10 may be fabricated from an array of material colors and/or designs, rather than conventional gauze color. It may also include one or more optional decorative patterns 20 for added uniqueness and likely increased satisfaction by the wearer.

The surgical sock 10 includes a forefoot region 11, a foot region 12, and an ankle region 13 terminating in an opening 14 into which the user places a bandaged foot (not shown). The identified regions of the sock 10 may be fabricated all of the same elasticity. Alternatively, the ankle region 13 and the opening 14 may have greater elasticity than the foot region 12 and the forefoot region 11 so that the bandaged foot may be inserted into the sock 10 and yet maintain a grasp on the ankle so that the sock 10 does not slip down below the ankle. The opening 14 may be formed with an additional elastic band 21 to complement the grasping capability inherent in the material used to make the sock 10. The sock 10 is configured so that the forefoot region 11 retains the toes of a bandaged foot therein when the foot is inserted completely into the sock 10.

In order to grip the foot properly yet minimize pressure on the wound and prevent patient discomfort, the forefoot region 11 has a greater diameter; that is, it is wider, than the ankle region 13 so that it fits snugly about the bandaged region of the foot without applying excess pressure and without bunching. Optionally, the foot region 12 may be sized comparable to that of the forefoot region 11, it may have the same sizing as that of the ankle region 13, or it may be a tapered region that acts as a transition between the dimensions of the forefront region 11 and the ankle region 13. Further, for procedures associated with the midfoot or rearfoot rather than the forefoot, the foot region 12 may actually be of greater dimensions than either the forefoot region 11 or the ankle region 13. A focus of the invention is to provide the surgical sock 10 with an expanded area where the bandage is located and a relatively narrower area where the ankle is located.

The surgical sock 10 may further optionally include one or more rubber bumps or pads 15 either bonded to the underside of the sock 10 in the forefoot region 11, the foot region 12, or both. Alternatively, the pads 15 may be incorporated into the fabric material associated with the fabrication of the sock 10. The pads 15 are anti-skid devices or anti-slip devices designed to minimize the possibility of a post-surgical fall.

While the invention has been described with reference to a particular example embodiment, it is intended to cover all modifications and equivalents as described by the following claims.

What is claimed is:

1. A surgical sock comprising:
a. an ankle region including an opening for receiving a bandaged foot therein;
b. a forefoot region within which the toes of the bandaged foot are retained; and
c. a foot region intermediate between said ankle region and said forefoot region, wherein the sock is configured such that a region thereof associated with a bandaged portion of the bandaged foot is of greater dimension than said ankle region.

2. The sock as claimed in claim 1 wherein said forefoot region is of greater diameter than said ankle region.

3. The sock as claimed in claim 1 wherein said foot region is of greater diameter than said ankle region.

4. The sock as claimed in claim 1 wherein said forefoot region and said foot region are of substantially equivalent diameter that is greater than a diameter of said ankle region.

5. The sock as claimed in claim 1 wherein said forefoot region is of greater diameter than said ankle region and said foot region provides an intermediate tapering diameter between said forefoot region and said ankle region.

6. The sock as claimed in claim 1 wherein said ankle region includes an elastic band.

7. The sock as claimed in claim 1 wherein said forefoot region and said foot region have a common underside and wherein said common underside includes one or more anti-slip devices.

8. The sock as claimed in claim 7 wherein said one or more anti-slip elements are one or more rubber pads.

9. The sock as claimed in claim 1 wherein said ankle region has more elasticity than said forefoot region.

10. The sock as claimed in claim 1 fabricated with a hydrophobic fiber.

11. The sock as claimed in claim 1 fabricated with a bacteriostatic fabric.

12. The sock as claimed in claim 1 wherein each of said forefoot region, said foot region, and said ankle region includes one or more printed patterns thereon.
